SEQUENCE LISTING (21 sequences)

SEQ ID NO: 1 - protein, 5 aa, Homo sapiens
NWGRI

SEQ ID NO: 2 - protein, 5 aa, Homo sapiens
KCILF

SEQ ID NO: 3 - protein, 5 aa, Homo sapiens
LLLLL

SEQ ID NO: 4 - protein, 5 aa, Homo sapiens
CCCCC

SEQ ID NO: 5 - protein, 5 aa, Homo sapiens
LCLLL

SEQ ID NO: 6 - protein, 5 aa, Homo sapiens
QRVAA

SEQ ID NO: 7 - protein, 5 aa, Homo sapiens
EFSGN

SEQ ID NO: 8 - protein, 5 aa, Homo sapiens
SSTLG

SEQ ID NO: 9 - protein, 5 aa, Homo sapiens
NWGRL

SEQ ID NO: 10 - protein, 4 aa, Homo sapiens
NWGR

SEQ ID NO: 11 - protein, 5 aa, Homo sapiens
SLWDQ

SEQ ID NO: 12 - protein, 7 aa, Homo sapiens
SFLTKGP

SEQ ID NO: 13 - protein, 22 aa, Homo sapiens
KTIIVQLNETVKINCIRPNNKT

SEQ ID NO: 14 - protein, 5 aa, Homo sapiens
FPSVE

SEQ ID NO: 15 - protein, 5 aa, Homo sapiens
WYFDV

SEQ ID NO: 16 - protein, 5 aa, Homo sapiens
WYYEV

SEQ ID NO: 17 - protein, 5 aa, Homo sapiens
HCPPG

SEQ ID NO: 18 - protein, 6 aa, Homo sapiens
DSDPSG

SEQ ID NO: 19 - protein, 6 aa, Homo sapiens
DSDPNG

SEQ ID NO: 20 - protein, 11 aa, Homo sapiens
KLVFFAEDVGS

SEQ ID NO: 21 - protein, 11 aa, Homo sapiens
RMPFYAEECAS

(19) United States
(12) Patent Application Publication
(10) Pub. No.: US 2004/0121398 A1
(43) Pub. Date: Jun. 24, 2004

(54) Method for predicting protein-protein interactions

(75) Inventors: Hirofumi Doi, Funabashi (JP); Atsushi Suzuki, Edogawa-ku (JP)

Correspondence Address: SUGHRUE MION, PLLC, 2100 PENNSYLVANIA AVENUE, N.W., SUITE 800, WASHINGTON, DC, 20037, US

(73) Assignees: DAIICHI PHARMACEUTICAL CO., LTD.; FUJITSU LIMITED

(21) Appl. No.: 10/237,673

(22) Filed: Sep. 10, 2002

Related U.S. Application Data
(63) Continuation-in-part of application No. PCT/JP01/01846, filed on Mar. 9, 2001.

(30) Foreign Application Priority Data
Oct. 3, 2000 (JP) JP2000-72485

(51) Int. Cl. G01N 33/53; G06F 19/00; G01N 33/48; G01N 33/50

(52) U.S. Cl. 435/7.1; 702/19

(57) ABSTRACT

The present invention relates to a method for predicting whether a protein or polypeptide (B) interacts with a specific protein or polypeptide (A), wherein the method comprises the steps of:

1) decomposing the amino acid sequence of protein or polypeptide (A) into a series of oligopeptides having a pre-determined length, by shifting, serially, by one amino acid residue from the N-terminal end to the C-terminal end of said protein or polypeptide (A);

2) searching, within a selected database of protein or polypeptide amino acid sequences, for a protein or polypeptide (C) comprising an amino acid sequence for each member of the series or for a protein or polypeptide (D) comprising an amino acid sequence homologous to an amino acid sequence for each member of the series, and detecting said protein or polypeptide (C) or said protein or polypeptide (D);

3) carrying out local amino acid sequence alignment between said protein or polypeptide (A) and the detected protein or polypeptide (C) or detected protein or polypeptide (D), whereupon if the local amino acid sequence alignment gives rise to any homology between the protein or polypeptide (A) and the detected protein or polypeptide (C) and/or protein or polypeptide (D), then;

4) determining the frequency of each member of said oligopeptides in a database of all proteins encoded by the organism, from which said protein or polypeptide (A) is derived, by calculating a product of the frequency of each amino acids composing said member of the oligopeptides in said database, and/or determining the local amino acid sequence alignment score on said oligopeptides so as to calculate the value by dividing the sum of the scores by the amino acid length of the protein or polypeptide (C) or protein or polypeptide (D), whereby said frequency of said oligopeptide and/or said value indicates that said protein or polypeptide (C) or protein or polypeptide (D) corresponds to said protein or polypeptide (B) which interacts with said protein or polypeptide (A); and to

a recording medium for carrying out the above method, a device comprising the recording medium, and proteins obtained thereby.

### FIELD OF THE INVENTION

The present invention relates to a method for predicting protein-protein interactions, a device therefor, and proteins obtained using the method and device.

### BACKGROUND OF THE INVENTION

Many proteins carry out their function by interacting with other proteins or the same protein. Thus, it is important to elucidate protein-protein interactions in the development of pharmaceuticals, the breeding in agriculture, and the like. Notably, along with the progress of genome analysis and cDNA analysis of various organisms including pathogenic microorganisms, the number of genes newly found and proteins encoded thereby whose functions are not known is rapidly increasing. Elucidating protein-protein interactions may permit one to predict the function of a protein whose function is not known.

A conventional method that has been used to screen for a protein interacting with a certain protein so as to elucidate their interactions, is the so-called two-hybrid system (Field, S. The two-hybrid system to detect protein-protein interaction. METHODS: A Companion to Meth. Enzymol., 5, 116-124, 1993). However, the two-hybrid system is a screening-based experiment, whose operation is complicated and time-consuming. Also, the number of proteins obtained is lower than that expected. In addition, this method has a disadvantage in that the results depend on the quality of the cDNA library used. In other words, this method has the risk that a gene encoding a protein interacting with a certain protein is not contained in the cDNA library used.

On the other hand, protein databases based on genome analysis and cDNA analysis have been enhanced, such that a method has also been adopted wherein a protein complex in a cell is subjected directly to MALDI-TOF mass spectrometry, followed by searching in the database for a fragment of the amino acid sequence thereof (Yates, JR 3rd, J. Mass Spectrom. 33, 1-19, 1998; Humphrey-Smith, I., et al., Electrophoresis, 18, 1217-1242; Kaufmann, R., 1995, J. Biotechnol., 41, 155-175, 1997). This method gives information concerning proteins that form a complex, but does not give any information concerning the protein-protein interaction. Thus, it must be experimentally confirmed which proteins interact with each other.

### SUMMARY OF THE INVENTION

In one embodiment, the present invention relates to a method for predicting whether a protein or polypeptide (B) interacts with a specific protein or polypeptide (A), wherein the method comprises the steps of:

1) decomposing the amino acid sequence of protein or polypeptide (A) into a series of oligopeptides having a pre-determined length, by shifting, serially, by one amino acid residue from the N-terminal end to the C-terminal end of said protein or polypeptide (A);

2) searching, within a selected database of protein or polypeptide amino acid sequences, for a protein or polypeptide (C) comprising an amino acid sequence for each member of the series or for a protein or polypeptide (D) comprising an amino acid sequence homologous to an amino acid sequence for each member of the series, and detecting said protein or polypeptide (C) or said protein or polypeptide (D);

3) carrying out local amino acid sequence alignment between said protein or polypeptide (A) and the detected protein or polypeptide (C) or detected protein or polypeptide (D), whereupon if the local amino acid sequence alignment gives rise to any homology between the protein or polypeptide (A) and the detected protein or polypeptide (C) and/or protein or polypeptide (D), then;

4) determining the frequency of each member of said oligopeptides in a database of all proteins encoded by the organism, from which said protein or polypeptide (A) is derived, by calculating a product of the frequency of each amino acids composing said member of the oligopeptides in said database, and/or determining the local amino acid sequence alignment score on said oligopeptides so as to calculate the value by dividing the sum of the scores by the amino acid length of the protein or polypeptide (C) or protein or polypeptide (D), whereby said frequency of said oligopeptide and/or said value indicates that said protein or polypeptide (C) or protein or polypeptide (D) corresponds to said protein or polypeptide (B) which interacts with said protein or polypeptide (A).

It is preferred that the length of the oligopeptide is 4-15 amino acids.

In addition, in another embodiment, the present invention relates to a recording medium carrying a program to enable a computer system to predict whether a protein or polypeptide (B) interacts with a specific protein or polypeptide (A), comprising at least the following means a) to f):

a) a means for inputting amino acid sequence information of the protein or polypeptide (A) and storing the information;

b) a means for decomposing the above-mentioned information into a series of oligopeptides as sequence information, and a means for storing the sequence information consequently obtained;

c) a means for storing an input protein database;

d) a means for accessing the stored protein database and detecting a protein or polypeptide (C) having an amino acid sequence of said oligopeptide or a protein or polypeptide (D) having an amino acid sequence homologous to the amino acid sequence of said oligopeptide, and a means for storing and calculating a detected result;

e) a means for carrying out local alignment between the protein or polypeptide (A) and the detected protein or polypeptide (C) or protein or polypeptide (D), and a means for storing and calculating a result; and

f) a means for obtaining a resultant value of a frequency of an amino acid and/or a frequency of said oligopeptide from a protein database, followed by showing an index for predicting protein-protein interactions from the resultant value and a resultant value of said local alignment, and a means for storing and displaying the result and consequently detecting protein or polypeptide (B) which interacts with the protein or polypeptide (A).

In a further embodiment, the present invention relates to a recording medium comprising at least one of the following means g) to 1) in addition to the means a) to f):

g) a means for ranking strength of protein-protein interactions among detected proteins or polypeptides (B) based on the indexes calculated from a resultant value of local alignment and a resultant value of a frequency of an amino acid and/or a frequency of an oligopeptide in a protein database in the case that more than one (B) exist that are detected, and a means for storing and displaying the result;

h) a means for displaying full-length of amino acid sequences of the protein or polypeptide (A) and the protein or polypeptide (B) that is detected, followed by indicating a location of partial sequence to be aligned in the full-length sequence in the case that amino acid partial sequences are aligned by local alignment between the protein or polypeptide (A) and the protein or polypeptide (B);

i) a means for calculating a stereo structure model in the case that a stereo structure of the protein or polypeptide (A) or the protein or polypeptide (B) that is detected is known or in the case that homology modeling enable to make a stereo structure model, followed by displaying the structure of the amino acid partial sequences that are aligned by local alignment between the protein or polypeptide (A) and the protein or polypeptide (B) on the stereo structure;

j) a means providing a function of classifying and storing proteins in a protein database to narrow a searching area;

k) a means for serially inputting each protein in a protein database as the protein or polypeptide (A); and

l) a means for storing a genome database.

In still another embodiment, the present invention relates to a device for predicting protein-protein interactions comprising the means that are carried by the above-mentioned recording medium.

In an additional embodiment, the present invention relates to a method for specifying proteins or polypeptides that interact with each other, which comprises identifying a protein or polypeptide (B) that is predicted to interact with a specific protein or polypeptide (A) by the above-mentioned prediction method or prediction device, and then experimentally confirming the presence of the interaction between the protein or polypeptide (A) and the protein or polypeptide (B).

Furthermore, in another embodiment, the present invention relates to a protein or polypeptide that is specified by the above method.

In still another embodiment, the present invention relates to a method of screening for a compound that is capable of controlling the interaction of a specific protein or polypeptide (A) with a protein or polypeptide (B) utilizing the above-mentioned prediction method or prediction device.

The method of screening mentioned above may comprises the steps of:

1) contacting the compound with the protein or polypeptide (A) and the protein or polypeptide (B) that are predicted to interact with each other using said prediction method or prediction device, and;

2) detecting the presence or absence or change of the interaction using the system for detecting the interaction of protein or polypeptide (A) with the protein or polypeptide (B).

In yet another embodiment, the present invention relates to a novel compound obtained by the screening method and a novel compound capable of controlling the interaction of the protein or polypeptide (A) with the protein or polypeptide (B) obtained by drug design based on information of the compound obtained.

In another embodiment, the present invention relates to an oligopeptide comprising amino acid sequence SEQ ID No: 1 which is capable of controlling the interaction of verotoxin 2 (VTII) with Bcl-2, or an oligopeptide that comprises an amino acid sequence homologous to the oligopeptide and is capable of controlling the interaction of VTII with Bcl-2, or a polypeptide that contains any of these oligopeptides and is capable of controlling the interaction of VTII with Bcl-2.

In addition, in one embodiment, the present invention, relates to a composition useful for inhibiting cell death comprising an oligopeptide comprising amino acid sequence SEQ ID NO: 1, and a pharmaceutically acceptable carrier or diluent.

In still another embodiment, the present invention, relates to a method of screening for a compound capable of controlling interaction of VTII with Bcl-2, wherein the method utilizes the above-mentioned oligopeptide.

The method of screening may comprise the steps of:

1) contacting the compound with VTII and the above-mentioned oligopeptide or with Bcl-2 and the oligopeptide, then;

2) detecting the presence or absence or change of the interaction of oligopeptide with VTII or Bcl-2 using the system for detecting the interaction.

In yet another embodiment, the present invention relates to a method of screening for a compound capable of controlling interaction of VTII with Bcl-2, wherein the method utilizes the above-mentioned polypeptide

The method of screening may comprise the steps of:

1) contacting the compound with VTII and the above-mentioned polypeptide or with Bcl-2 and the polypeptide, then;

2) detecting the presence or absence or change of the interaction of polypeptide with VTII or Bcl-2 using the system for detecting the interaction.

In a further embodiment, the present invention relates to a method for determining a sequence of an oligonucleotide coding an oligopeptide involved in interaction of a specific protein or polypeptide (A) with a protein or polypeptide (B) that is predicted to interact with the protein or polypeptide (A), wherein the method uses the above-mentioned prediction method or the above-mentioned prediction device.

In another embodiment, the present invention relates to a series of combinations of human proteins obtained by the above-mentioned prediction method or the above-mentioned prediction device.

In addition, in an embodiment, the present invention relates to a method for selecting a combination of proteins having a protein-protein interaction that is related to a disease, wherein the method comprises selecting the combination based on the information of a known protein that is related to the disease from the above-mentioned series of combination of proteins.

Further in another embodiment, the present invention relates to a series of combinations of proteins having protein-protein interaction that are related to diseases, and which are obtained by the above-mentioned method.

In yet another embodiment, the present invention relates to a method of screening for a compound that controls the interaction of a certain combination and/or two proteins further selected from the series of combinations of proteins having a protein-protein interaction that are related to diseases obtained as mentioned above.

In a still further embodiment, the present invention relates to a compound obtained by the method of screening a compound which controls the interaction.

In yet another embodiment, the present invention relates to a method for predicting a processing site of a protein by predicting the protein-protein interaction of a specific protein with an enzyme cleaving said protein using the above-mentioned prediction method or device.

In addition, in one embodiment, the present invention relates to an amino acid sequence that contains a protein-processing site obtained by the above-mentioned prediction method for a protein-processing site, and/or an amino acid sequence that contains a partial sequence homologous to the processing site.

### BRIEF DESCRIPTION OF DRAWINGS

FIG. 1a illustrates 20 amino acid residues from the amino terminal end of verotoxin 2.

FIG. 1b illustrates oligopeptides, each having an amino acid sequence length of 5 residues, which were obtained by decomposing the amino acid sequence of FIG. 1a using a computer program.

FIG. 1c illustrates oligopeptides, each having an amino acid sequence length of 6 residues, which were obtained by decomposing the amino acid sequence of FIG. 1a using a computer program.

FIG. 2 illustrates oligopeptides, each having an amino acid sequence length of 5 residues, which were obtained by decomposing the 13 residues from the amino terminal end of verotoxin 2 using a computer program, and human proteins comprising the amino acid sequence of the oligopeptides.

FIG. 3 illustrates the result of local alignment whereby oligopeptides that comprise a portion of verotoxin 2 (VTII) and human β-adrenergic receptor kinase 2 (ARK2) were obtained.

FIG. 4a illustrates the frequency of each of the 20 kinds of amino acids in Escherichia coli proteins.

FIG. 4b illustrates the percentage that each of the 20 kinds of amino acid is present in Escherichia coli proteins.

FIG. 5 is a simplified flow diagram illustrating a computer supported method for prediction according to one aspect of the present invention.

FIG. 6 illustrates amino acid sequences of oligopeptides derived from verotoxin 2 which are also present in human proteins that are related to cell death, and the corresponding human proteins.

FIG. 7 illustrates the result of local alignment whereby oligopeptides that comprise a portion of verotoxin 2 (VTII) and Bcl-2, were obtained.

FIG. 8 illustrates the result of local alignment whereby oligopeptides that comprise a portion of verotoxin 2 (VTII) and Bcl-xL, were obtained.

FIG. 9 illustrates the result of local alignment whereby oligopeptides that comprise a portion of verotoxin 2 (VTII) and MCL-1, were obtained.

FIGS. 10a-b illustrate the result of local alignment whereby oliopeptides that comprise a portion of verotoxin 1 (VTI) and Bcl-2 (FIG. 10a) or Bcl-xL (FIG. 10b), were obtained.

FIGS. 11a-b illustrate electrophoretic patterns showing the result of confirmational experiments using HepG2 cells and B10 cells showing that verotoxin 2 (VTII) and Bcl-2 interact with each other ((FIG. 11a) and (FIG. 11b) right), but that verotoxin 1 (VTI) and Bcl-2 do not interact with each other ((FIG. 11b) left). In the figures, Bcl-2 IPs and VTII IPs indicate that Bcl-2 and VTII were immunoprecipitated by anti-Bcl-2 antibody and anti-VTII antibody, respectively. FIG. 11a (left) illustrates the result of western blotting using anti-Bcl-2 antibody (Bcl-2 WB) FIG. 11a (right) illustrates the result of western blotting using anti-VTII antibody (VTII WB). FIG. 11b illustrates electrophoretic patterns showing the result of confirmation of the subcellular fraction of B10 cells that were treated with verotoxin 1 (VTI) (left) or verotoxin 2 (VTII) (right), where these proteins were detected using anti-VTI antibody and anti-VTII antibody, respectively.

FIG. 12 illustrates the sites which correspond to the local alignment of verotoxin 2 (VTII) and Bcl-2.

FIG. 13 illustrates, using a wire model, the portion (in bold) that is homologous to the partial sequence of verotoxin 2 (VTII) on the stereo structure of Bcl-xL.

FIG. 14 illustrates, using a wire model, the portion (in bold) that is homologous to the partial amino acid sequence of Bcl-xL on the stereo structure of verotoxin 2.

FIG. 15 illustrates that oligopeptide NWGRI (SEQ ID NO: 1) which comprises a portion of verotoxin 2 (VTII) and Bcl-2, suppresses cell death induced by VTII in a dose dependent manner of NWGRI.

FIGS. 16a-b illustrate the result of local alignment whereby oligopeptides that comprise a portion of human helper T cell surface protein CD4 and HIV-1 virus surface protein gp120, were obtained. FIG. 16a illustrates oligopeptides that comprise a portion of CD4 and gp120. FIG. 16b illustrates amino acid sequences of a region having a high local homology in CD4 and gp120.

FIG. 17 illustrates the result of local alignment whereby oligopeptides that comprise a portion of CED-4 (a cell death-related protein of nematode) and MAC-1 protein (which binds to CED-4), were obtained.

FIG. 18 illustrates the result of local alignment whereby oligopeptides that comprise a portion of amyloid precursor protein (APP) and BASE (an enzyme which cleaves the protein), were obtained.

FIGS. 19a-b illustrate the result of local alignment whereby oligopeptides that comprise a portion of furin-precursor protein (furin-pre) and von Willebrand factor precursor protein (VWF-pre), were obtained. FIG. 19a illustrates oligopeptides that comprise a portion of both proteins. FIG. 19b illustrates an amino acid sequence of a region having a high local homology in both proteins.

FIG. 20 illustrates the result of local alignment whereby oligopeptides that comprise a portion of amyloid precursor protein (APP) and protein PC7 (which is considered to be involved in the processing thereof), were obtained. In the figure, symbol “=” indicates a site that is predicted to be a cleavage site.

### BRIEF DESCRIPTION OF REFERENCE NUMERALS

a Means for inputting

b Means for decomposing in a series of oligopeptides and storing the same as sequence information

c Means for storing as sequence information

d Means for searching and storing as sequence information

e Means for carrying out local alignment and storing as sequence information

f Frequency-calculating/memory-displaying means

g Ranking/memory-displaying means

h Location-displaying means

i Stereo structure-calculating/memory-displaying means

j Means for classifying proteins and storing as sequence information

k Sequentially inputting means

l Means for storing as sequence information

m Keyboard

n Controlling means

o Outputting means

### DETAILED DESCRIPTION OF THE INVENTION

The embodiments of the present invention will be described in more detail below as well as the principle and method of the present invention, a recording medium that carries a program for carrying out the method, a device that works the function, proteins and polypeptides that are obtained by the method and device. The following description is given only for illustration, and it not intended to limit the present invention.

Technical and scientific terms used in the specification have the meanings usually understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties.

In one embodiment, the present invention relates to a method for predicting protein-protein interactions, which is based on the following idea: protein is composed of a sequence consisting of 20 kinds of amino acids, but these amino acids are not randomly placed. Therefore, it is considered that an oligopeptide that is a partial sequence of a protein has a role in a species of a living organism.

For example, an oligopeptide that is a part of a certain enzyme is considered to play a role in recognizing a substrate. In another protein, an oligopeptide that plays an important role in interacting with other proteins is considered to exist. In this way, it is necessary to consider the function or interaction of a protein from the oligopeptide level. In addition, the frequency of the appearance of certain oligopeptides in all of the proteins encoded by the genome in one organism is not even. Some oligopeptides frequently occur in various proteins; others do so only rarely. It is very likely that an oligopeptide that occurs with low frequency is an oligopeptide that is unique to each protein. Such an oligopeptide might determine the feature or function of the protein.

On the other hand, the fact that proteins interact with each other implies that the interacting proteins perform a function in cooperation with each other whereby the organism carries on its biotical activity. If it is assumed that one oligopeptide corresponds to one function, two proteins that interact with each other might have the same oligopeptide or homologous oligopeptides. In addition, these two proteins might have homologous sequence structure in a part other than the oligopeptide that is the same.

As one of the techniques of similarity search for analyzing homology of two proteins, a method of comparison by aligning the primary structures of both proteins is known (Minoru Kanahisa “Introduction to genome informatics (in Japanese)” Kyoritsu Publishing Co., Ltd., 93-104, 1996). This sequence alignment includes ‘global alignment’ and ‘local alignment’. The ‘global alignment’ comprises aligning the entire sequences, and the ‘local alignment’ comprises locally aligning only homologous parts extracted from their sequences. In any alignment, the alignment is carried out so that the relation between/among two sequences or more can be as clear as possible. Many combinations exist in the alignment depending on the length of the sequence. Methods for carrying out combinatorial optimization include the dynamic programming method. The Smith-Waterman method (Smith, T F and Waterman, M S, J. Mol. Biol. 147, 195-197, 1981) that is based on the principle that dynamic programming gives an estimation function on the combinatorial optimization of sequences. A value of the estimation function, i.e., ‘homology score’ or ‘score’ permits estimating homology between two proteins. It can be estimated that the higher the score between proteins that were compared, the higher the homology between these proteins. As for the local alignment, it is carried out by setting a threshold to the score, followed by carrying out combinatorial optimization of partial sequences, when the combination of sequences is searched by dynamic programming (e.g., Gotoh, O., Pattern matching of biological sequences with limited storage, Comput. Appl. Biosci. 3, 17-20, 1987). The local alignment method permits searching the homologous structure in a part of the protein other than where the oligopeptide that is the same portion to two proteins is located.

Protein-protein interactions might have been conserved in the process of evolution. The case of verotoxin of Escherichia coli and Bcl-2, as described later, implies that one function has been conserved in the protein-protein interaction beyond species, whence structurally similar amino acid sequences might exist. In addition, in the processing of amyloid precursor protein and von Willebrand Factor (VWF) precursor protein, as described later, the function of proteolysis might have been conserved in the protein-protein interaction, and structurally similar amino acid sequences might exist.

A method for predicting protein-protein interactions that is created based on the above idea may also permit predicting a network of functions that has been known only as a single function in the past and describing a new image of life based on the results that were predicted on a computer and on the overall relation of actions that is different from the image of life being reached by the accumulation of facts that have been obtained by the enumeration principle of molecular biology.

In addition, if the prediction for interactions is possible not in one organism but between two organisms, e.g., human beings and pathogenic microorganisms, the elucidation of the pathogenesis that has not been known so far might become possible.

Concretely, one embodiment of the above method for predicting protein-protein interactions is a method of extracting and predicting a counter-protein from a protein database and the like, which interacts with a protein that was obtained by genome analysis or cDNA analysis whose function is unknown or a protein whose function is known, wherein the method comprises, for example, the following steps 1-4:

In step 1, an amino acid sequence of a specific protein or polypeptide is decomposed into a series of oligopeptides having a pre-determined length as the sequence information. In step 2, proteins or polypeptides are determined which comprise each oligopeptide. In step 3, homology of partial structures between the proteins is estimated by local alignment. In step 4, each oligopeptide is further evaluated by a frequency of occurrence.

Each step will be described below more in detail:

Step 1:

The amino acid sequence of a specific protein or polypeptide (A), such as a protein or polypeptide that is obtained by genome analysis or cDNA analysis and whose function is not known or a protein or polypeptide whose function is known, is decomposed into oligopeptides as sequence information by shifting, serially, by one amino acid residue from the amino terminal end to the carboxyl end.

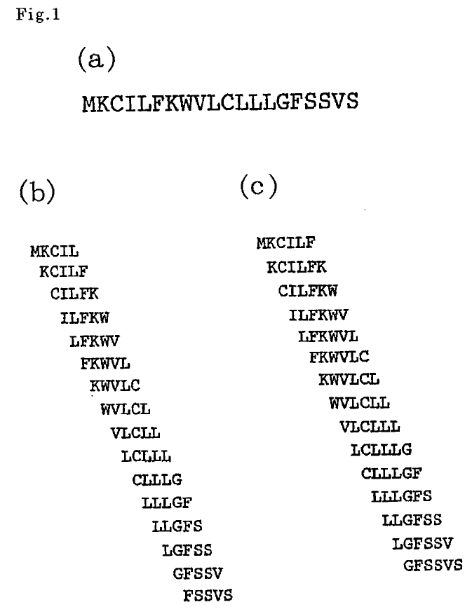

For example, FIGS. 1a-b illustrate oligopeptides (FIG. 1b) having an amino acid length of 5 residues that were obtained by decomposing the first 20 residues (FIG. 1a) of verotoxin 2 (VTII) of Escherichia coli 0157:H7 from its amino terminal end as sequence information. Amino acids and oligopeptides are given in their one-letter symbols hereafter.

When step 1 is carried out, the amino acid length of oligopeptide that is decomposed as the sequence information is preferably 4 to 15 residues, more preferably 4 to 8 residues. The longer the length of an oligopeptide, the greater the particularity of the oligopeptide, as shown in Example 2.

Step 2:

In step 2, a protein or polypeptide (C) comprising an amino acid sequence of an oligopeptide that was obtained by the decomposition in step 1 or a protein or polypeptide (D) having an amino acid sequence that is homologous to the oligopeptide is searched for in an amino acid sequence database of proteins or polypeptides. The number of detected proteins or polypeptides (C) or (D) can be large or can be one depending on the oligopeptide used.

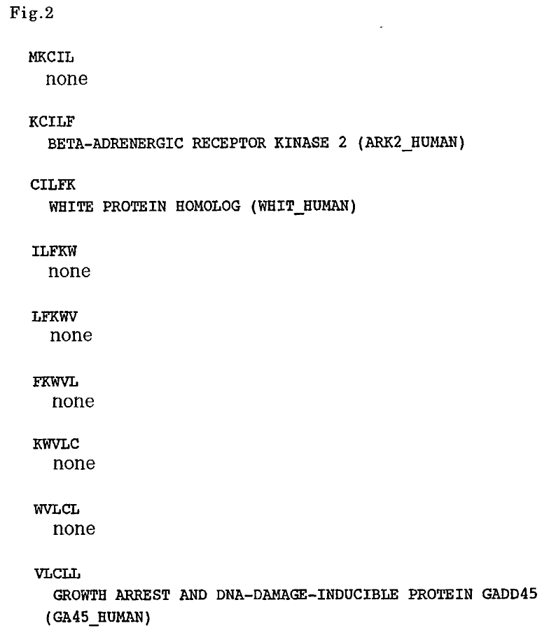

For example, FIG. 2 illustrates the results of searching for proteins having 9 oligopeptides each consisting of 5 amino acids obtained by decomposing 13 residues of verotoxin 2 (VTII) of Escherichia coli 0157:H7 from the amino terminal end, in a protein database (SWISS-PROT version 35). Verotoxin 2 causes food poisoning and/or renal damage in human beings, so that human proteins can be targets for searching for counter-proteins that interact with VTII. For example, such a search shows that a human protein comprising oligopeptide KCILF (SEQ ID NO: 2) shown in FIG. 2 (second) is β-adrenergic receptor kinase 2 (ARK2 HUMAN).

Step 3:

Local alignment is carried out between the above protein or polypeptide (A) and the protein or polypeptide (C) or protein or polypeptide (D) that is obtained in the search in Step 2.

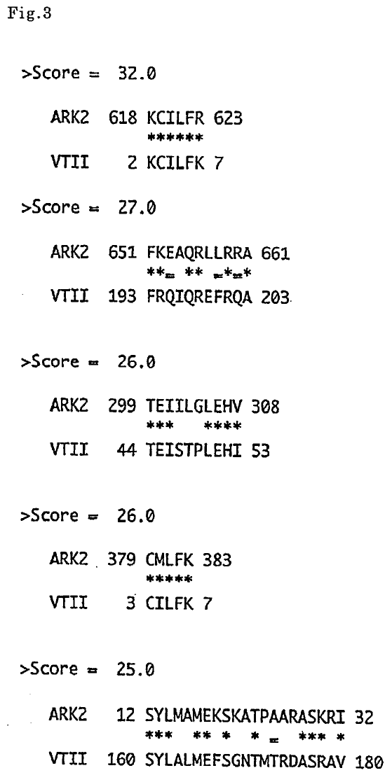

For example, FIG. 3 illustrates the result of local alignment between verotoxin 2 (VTII) and β-adrenergic receptor kinase 2 (ARK2).

Step 4:

If the result of the local alignment in step 3 shows any homology of partial sequence between the above protein or polypeptide (A) and the detected protein or polypeptide (C) and/or protein or polypeptide (D), the protein or polypeptide (C) and/or protein or polypeptide (D) are/is predicted to possibly be a protein or polypeptide (B) that interacts with protein or polypeptide (A). Moreover, the frequency of amino acid(s) and/or the frequency of oligopeptide that is present in both protein or polypeptide (A) and the detected protein or polypeptide (C) and/or protein or polypeptide (D) is calculated from a protein database, followed by evaluating the particularity of each oligopeptide in the protein database or in the genome of an organism having the protein or polypeptide (A) or the detected protein or polypeptide (C) and/or protein or polypeptide (D). If the particularity is high, the reliability of the prediction is evaluated to be high that the protein or polypeptide (C) and/or protein or polypeptide (D) are/is a protein or polypeptide (B) that interacts with the protein or polypeptide (A).

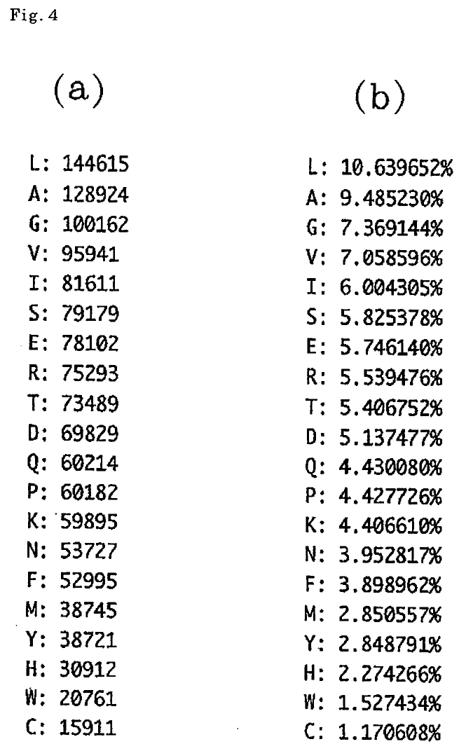

For example, an index of particularity of the above oligopeptide KCILF (SEQ ID NO: 2) is calculated to be 1284.86×10⁻¹⁰ from the composition ratio (FIG. 4b) that is calculated from the frequency of amino acid (FIG. 4a) in all of the proteins encoded by the genome of Escherichia coli shown in FIG. 4, so that the particularity is high. An oligopeptide consisting of 5 amino acids that is calculated to have low particularity in the E. coli genome is LLLLL (SEQ ID NO: 3), i.e., the particularity index is 136344.34×10⁻¹⁰. An oligopeptide consisting of 5 amino acids that is calculated to have the highest particularity is CCCCC (SEQ ID NO: 4), with a particularity index of 2.208×10⁻¹⁰, but the oligopeptide is not found in the E. coli genome. Therefore, the prediction that verotoxin 2 interacts with β-adrenergic receptor kinase 2 is evaluated to have high reliability from the value of the particularity index.

In order to confirm further the protein-protein interaction, the gene encoding the protein may be cloned for expression based on the information of the obtained proteins that interact with each other. For example, as described in the examples later, VTII and Bcl-2 were predicted to interact with each other by the above predicting method or an predicting device carrying a program for the predicting method. This could be confirmed by experiments wherein cells in which Bcl-2 is expressed and cells in which Bcl-2 is not expressed were treated with VTII, followed by co-immunoprecipitating with anti-Bcl-2 antibody and anti-VTII antibody. In addition, it is possible to specify the oligopeptide as an important interacting site, for example, by introducing a mutation by a well-known method into the amino acid sequence of an oligopeptide that is predicted to be an interacting site, followed by confirming that the interaction is lost. The method for confirming interactions experimentally is not limited to the above ones, but any of techniques that are applicable by those skilled in the art may be used.

In addition, if it is confirmed that an oligopeptide, which was predicted to be an interacting site, interrupts a protein-protein interaction and suppresses any function or action of the protein, then such an oligopeptide can be utilized as an agent for suppressing the action of the protein. For example, as described in an example below, oligopeptide NWGRI (SEQ ID NO: 1), which was predicted to be the interacting site for VTII and Bcl-2, suppresses cell death induced by VTII and can be used as an agent against cell death. Such a low-molecular-weight compound can be utilized as pharmaceuticals, reagents, and the like.

One embodiment of the invention resides in a computer program product for predicting protein-protein interactions. Prior to describing the specifics of this embodiment, the broad meaning of some of the terms to be used will be discussed.

As used herein, the term “computer system” is to be understood to include at least a memory and a processor. In general, the memory will store, at one time or another, at least portions of an executable program code, and the processor will execute one or more of the instructions included in that executable program code. It will be appreciated that the term “executable program code” and the term “software” mean substantially the same thing for the purposes of this description. It is not necessary to the practice of this invention that the memory and the processor be physically located in the same place. That is to say, it is foreseen that the processor and the memory might be in different physical pieces of equipment.

On a practical level, the software that enables the computer system to perform the operations described further below in detail, may be supplied on any one of a variety of media. Furthermore, the actual implementation of the approach and operations of the invention may begin with actual statements written in a programming language. Such programming language statements, when executed by a computer, cause the computer to act in accordance with the particular content of the statements. Furthermore, the software that enables a computer system to act in accordance with the invention may be provided in any number of forms including, but not limited to, original source code, assembly code, object code, machine language, compressed or encrypted versions of the foregoing, and any and all equivalents.

One familiar with the field of computers will appreciate that “media”, or “computer-readable media”, or “recording medium” as used herein may include a diskette, a tape, a compact disc, an integrated circuit, a ROM, a CD, a cartridge of any shape (such as a memory stick or even a portable key-shaped memory), a remote transmission via a communications circuit, or any other similar medium useable by computers. For example, to supply software for enabling a computer system to operate in accordance with the invention, the supplier might provide a diskette or might transmit the software in some form via satellite transmission, via a direct telephone link, or via the Internet. Thus, the term, “recording medium” is intended to include all of the foregoing and any other medium by which software may be provided to a computer.

Although the enabling software (referred to in general terms as a “program”) might be “written on” a diskette, “stored in” an integrated circuit, or “carried over” a communications circuit, it will be appreciated that, for the purposes of this application, the program will be referred to as being “carried” by the recording medium. Thus, the concept of “carrying” is intended to encompass the above and all equivalent ways in which a program is associated with a recording medium.

One familiar with software understands that the functionality of the software can be characterized in various ways. For example, one function may be thought of as a module, a routine, a subroutine, a program, a unit of code, an object, a library, or the like. For the sake of generality, however, the term “means” may be used herein. When used in connection with a discussion of a recording medium or device, the term “means” may be understood to relate to a function of software regardless of the actual implementation as a unit, object, routine, or the like.

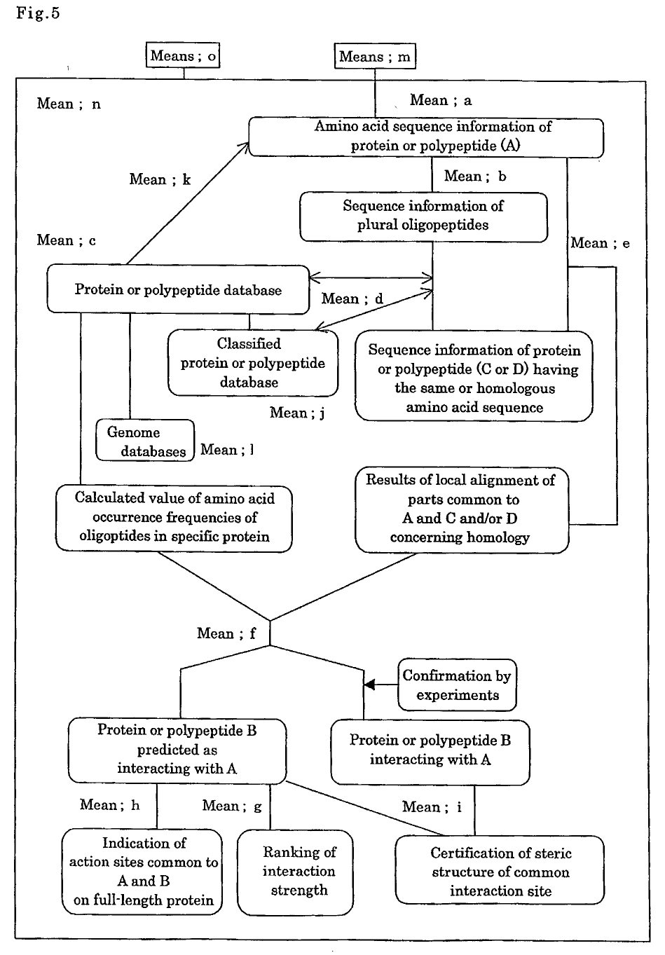

Next, a recording medium and device that carry a program for the above method for predicting protein-protein interactions will be described. The above recording medium and device comprise at least the following means (a) to (f). FIG. 5 illustrates an example of the constitution.

Inputting Means (a):

A means for inputting the amino acid sequence information concerning a specific protein or polypeptide (A) such as a protein or polypeptide, which was obtained by genome analysis or cDNA analysis, whose function is not known or protein or polypeptide whose function is known.

Means for Decomposing into a Series of Oligopeptides and Storing Sequence Information (b):

A means for decomposing the amino acid sequence information that was input by inputting means (a) into a series of oligopeptides having a pre-determined length as sequence information by shifting, serially, by one amino acid residue from the amino terminal end to the carboxyl end, and storing the result.

Storing Means (c):

A means for storing a database that was input in concerning with a protein or polypeptide.

Searching/Storing Means (d):

A means for accessing a database concerning a protein or polypeptide that is stored in storing means (c), followed by searching for a protein or polypeptide (C) comprising the amino acid sequence of the above oligopeptide or a protein or polypeptide (D) comprising an amino acid sequence that is homologous to the amino acid sequence of the above oligopeptide, and storing the result.

Carrying Out Local Alignment/Storing Means (e):

A means for carrying out local alignment between the above protein or polypeptide (A) and the detected protein or polypeptide (C) and/or (D), and storing the result.

Frequency-Calculating/Memory-Displaying Means (f):

A means for calculating an index for predicting protein-protein interactions from the result of the above local alignment and the result obtained after calculating a frequency of an amino acid and/or a frequency of an oligopeptide in a peptide or polypeptide database, and storing and displaying the result.

In addition, in the above program for predicting protein-protein interaction, it is also possible to comprise the following means (g) to (l) in an appropriate combination:

Ranking/Memory-Displaying Means (g):

A means having a function of ranking proteins or polypeptides (B), when more than one protein or polypeptide (B) is detected, by using the result of the local alignment and the result of the calculation of a frequency of an amino acid and/or a frequency of an oligopeptide from a protein database as indexes and a function of storing/displaying the result.

Location-Indicating Means (h):

A means for displaying full-length amino acid sequences of the protein or polypeptide (A) and the protein or polypeptide (B) followed by indicating a location of partial sequence to be aligned in the full-length sequences in the case that amino acid partial sequences are aligned between the protein or polypeptide (A) and the detected protein or polypeptide (B)

Stereo Structure-Calculating/Memory-Displaying Means (i):

A means for calculating a stereo structure model followed by displaying the structure of the amino acid partial sequences that are aligned between the protein or polypeptide (A) and the protein or polypeptide (B) on the stereo structure in the case that a stereo structure of the protein or polypeptide (A) or the protein or polypeptide (B) that is detected is known or in the case that a stereo structure model can be constructed by homology modeling.

Protein-Classifying/Storing Means (j):

A means having a function of classifying proteins or polypeptides in a protein or polypeptide database by feature, function, and/or origin to narrow a searching area followed by storing them.

Sequentially Inputting Means (k):

A means for sequentially inputting each protein or polypeptide in a protein or polypeptide database as the protein or polypeptide (A).

Storing Means (l):

A means having a function of storing a genome database.

The above means are carried on an appropriate medium.

As one embodiment of use, each of these means may be provided as a device containing a recording medium selectively carrying it as a program. A device for predicting protein-protein interactions is operated as described below (see FIG. 5).

A specific protein or polypeptide (A), such as a protein or peptide which was input by an inputting means (a) and whose function is unknown or known, is decomposed by means for decomposing into a series of oligopeptides and storing sequence information (b) into a series of oligopeptides having a pre-determined length as sequence information, and the oligopeptides are stored. In this case, the protein or polypeptide (A) is sequentially input by a sequentially inputting means (k) from a means (c) that stores a database that was input concerning the protein or polypeptide, when desired. A search is carried out through a means (c) for the amino acid sequence of the above-stored oligopeptide by a searching/storing means (d), and a protein or polypeptide (C) comprising the amino acid sequence of the oligopeptide or a protein or polypeptide (D) comprising an amino acid sequence that is homologous to the amino acid sequence of the oligopeptide is detected and stored. When searching, it is also possible to classify proteins or polypeptides in a database to narrow the searching area by a protein-classifying/storing means (j), followed by searching within the resultant area. The above protein or polypeptide (A) and the detected protein or polypeptide (C) or (D) are subjected to local alignment by a locally aligning/memory-displaying means (e), and the result is stored. Next, by a frequency-calculating/storing means (f), the frequency of an amino acid and/or the frequency of the oligopeptide are/is calculated from a database that was stored on a means (c), and an index for predicting protein-protein interactions is calculated from the result and the above-obtained result of the local alignment and stored. Then, those obtained are displayed on the screen of the device, which are the protein or polypeptide (C) or (D) that are predicted to interact with the above protein or polypeptide (A), an amino acid sequence of an oligopeptide that is the same in these proteins, a frequency of the oligopeptide, indexes for predicting protein-protein interactions, and so on. Displayed results permits giving a protein or polypeptide (B) that has interaction with the above protein or polypeptide (A) based on the indexes for predicting protein-protein interactions. In addition, concerning the above protein or polypeptide (B), it is also possible to display the functional information of a protein that is stored on a means (c) and the gene information from a means (l) equipped when desired that stores a genome database. When more than one protein or polypeptide (B) is detected, a ranking/memory-displaying means (g) permits ranking the protein or polypeptide (B) in order of the particularity to interact with the above protein or polypeptide (A). It is also possible to indicate by a location-indicating means (h) which part of the full-length amino acid sequences of the above protein or polypeptide (A) and the detected protein or polypeptide (B) is the partial amino acid sequence that is aligned between the protein or polypeptide (A) and the protein or polypeptide (B). In addition, it is also possible to display a stereo structure of the protein or polypeptide (A) and the protein or polypeptide (B), as well as the part that is aligned between the protein or polypeptide (A) and the protein or polypeptide (B) by a stereo structure-calculating/memory-displaying means (i). This device may be equipped with keyboard (m), controlling means (n), outputting means (o), as shown also in FIG. 5, and so on as well as these means (a)-(l).

The above method for predicting protein-protein interactions or the above prediction device can further be used for screening a novel compound that controls the interaction of a specific protein or polypeptide (A) with a protein or polypeptide (B). The above method of screening a novel compound that controls the interaction of a specific protein or polypeptide (A) with a protein or polypeptide (B) is carried out based on the information of the amino acid sequence of a key oligopeptide. An amino acid sequence of a selected oligopeptide, an amino acid sequence of an oligopeptide homologous thereto, or a polypeptide comprising the amino acid sequence or the homologous amino acid sequence per se can be capable of controlling the interaction of the protein or polypeptide (A) with the protein or polypeptide (B). For example, in the case that the protein or polypeptide (B) having a receptor function to the protein or polypeptide (A) is in existence, it is likely that an oligopeptide that is screened by the above technique is antagonistic to the interaction of the protein or polypeptide (A) with the protein or polypeptide (B). For example, in the case that the protein or polypeptide (A) is activated by the interaction with the protein or polypeptide (B), it is likely that an oligopeptide that is screened by the above technique has a function as an agonist.

Concretely, as described in detail in the examples below, it was experimentally confirmed that an oligopeptide NWGRI described in SEQ ID NO: 1, which comprises a portion of VTII and Bcl-2, that were predicted and experimentally confirmed to interact with each other by the present invention, interrupts complex formation due to the interaction of VTII with Bcl-2, and suppresses cell death induced by VTII. Therefore, NWGRI oligopeptide can be used as a medicament for controlling a disease related to cell death induced by VTII, for example as a medicament for treating a disease caused by Escherichia coli O157 expressing VTII, more concretely as an agent against cell death. Moreover, an oligopeptide having an amino acid sequence homologous to the amino acid sequence and capable of controlling the interaction of VTII with Bcl-2, or a polypeptide comprising the amino acid sequence or an amino acid sequence homologous to the amino acid sequence and capable of controlling the interaction of VTII with Bcl-2, can also be used as a medicament for controlling a disease related to cell death induced by VTII. In addition, a novel compound capable of controlling the interaction of VTII with Bcl-2 can be obtained utilizing these oligopeptides and polypeptides by the drug design method or by applying of a known screening method.

In this way, a novel compound, which is obtained by drug design based on the information of an oligopeptide that is obtained by the above screening method according to an embodiment of the present invention, is capable of controlling the interaction of a specific protein or polypeptide (A) with a protein or polypeptide (B). Namely, to predict interaction of the above protein or polypeptide (A) with the above protein or polypeptide (B) permits one to make a derivative of the oligopeptide obtained by the above screening method and a low-molecular-weight compound having a structure homologous to the oligopeptide by a well-known drug design technique.

The above prediction method is also very useful for a method for determining the sequence of the oligonucleotide encoding an oligopeptide involved in interaction of a specific protein or polypeptide (A) with a protein or polypeptide (B). Applying well-known methods such as substitution, deletion, addition, insertion, or induced mutation based on this information permits one to obtain a useful oligonucleotide. The obtained oligonucleotide can be used for obtaining a compound for controlling the interaction of protein or polypeptide (A) with protein or polypeptide (B) on a gene level. For example, it is utilized for making an antisense oligonucleotide to interrupt the protein-protein interaction. In addition, the obtained oligonucleotide can be used for diagnosing a disease that is related to the protein-protein interaction.

In another embodiment, the present invention relates to a series of combinations of human proteins that are predicted to have protein-protein interactions, which are predicted by the above method or device for predicting protein-protein interaction. The series of combination of proteins can be provided as a catalogue or as a database. A series of combination of proteins which interact with each other that are involved in a disease can be obtained by selecting ones having protein-protein interactions that are related to a disease based on the information of known proteins that can be related to the disease from the series of combination of proteins having protein-protein interactions. These can be provided as a catalogue or as a database. These combinations of proteins are useful as a medicament for treating or preventing diseases or as ways to obtain medicaments. For example, a compound capable of controlling interaction of two proteins can be obtained by screening using a well-known screening method and by utilizing a combination of proteins that is obtained.

In the case that among combinations consisting of two proteins having a protein-protein interaction, one protein is an enzyme capable of processing protein and cleaves the other protein, the processing site of the protein that is cleaved can be predicted by the above method or device for predicting protein-protein interactions.

For example, as shown in an example below, prediction could be accomplished on the interaction of an amyloid precursor protein with an enzyme that is involved in its processing, and on the interaction of von Willebrand factor precursor protein with an enzyme furin that is involved in its processing. Namely, the above method or device for predicting protein-protein interactions permits predicting a cleavage site when a precursor protein is cleaved to act as a mature protein. In this way, a hitherto-unknown enzyme having a protein-processing action related to a disease and a protein that is cleaved by the enzyme can be obtained by predicting protein-protein interactions.

### EXAMPLES

Although advantages, features, and possible applications of the present invention are described below in greater detail with reference to exemplary embodiments, the present invention is not limited to the following examples. In addition, although SWISS-PROT version 35 was used as a protein database in the following examples, other protein databases or the like can also be used.

### Example 1

FIGS. 1a-c illustrate oligopeptides that were decomposed from the first 20 residues (FIG. 1a) of verotoxin 2 (VTII) of Escherichia coli O157: H7 from the amino terminal end, where the oligopeptides have an amino acid sequence length of 5 resides (FIG. 1b) as an example of step 1. FIG. 1c illustrates oligopeptides that were decomposed from the first 20 residues of verotoxin 2 (VTII) from the amino terminal end, where the oligopeptides have an amino acid sequence length of 6 residues.

### Example 2

In step 4 of the above method for predicting protein-protein interactions, values are used as an index for predicting the interaction of proteins or polypeptides. The values are calculated from the frequency of an amino acid in a protein or polypeptide database and the frequency of an oligopeptide in the protein or polypeptide database. By way of example, the particularity of oligopeptides is calculated from the frequency of the 20 kinds of amino acids in all of the proteins encoded by the genome of Escherichia coli shown in FIG. 4a. The percentage ‘Ai’ of each amino acid ‘ai’ in the database can be calculated to be as shown in FIG. 4b from the frequency of occurrence (FIG. 4a) of the 20 amino acids in all of the proteins encoded by the genome of Escherichia coli.

The particularity of oligopeptide a1a2a3a4a5 is calculated to be A1×A2×A3×A4×A5. For example, in the case of oligopeptide KCILF (SEQ ID NO: 2), it is calculated to be 4.406610×1.170608×6.004305×10.639652×3.898962×10⁻¹⁰. The particularity of oligopeptide LLLLL (SEQ ID NO: 3) is calculated to be 136344.34×10⁻¹⁰, and the particularity of oligopeptide CCCCC (SEQ ID NO: 4) is calculated to be 2.20×10⁻¹⁰.

The smaller the value, the greater the particularity of the oligopeptide. The oligopeptide that has the highest particularity among those having an amino acid sequence length of 5 residues is oligopeptide CCCCC (SEQ ID NO: 4), but this oligopeptide does not occur in any of the proteins encoded by the genome of Escherichia coli. In contrast, the oligopeptide that has the lowest particularity is oligopeptide LLLLL (SEQ ID NO: 3).

When a protein or polypeptide (A) is decomposed into oligopeptides in step 1, the longer the oligopeptide is, the greater the particularity of the oligopeptide.

### Example 3

In step 4 of the above method for predicting protein-protein interactions, as an index for predicting the interaction of proteins or polypeptides, the result of the local alignment is used. Here is mentioned an example in which scores of the alignment of a partial sequence by Gotoh's method (Gotoh, O., Pattern matching of biological sequences with limited storage, Comput. Appl. Biosci. 3, 17-20, 1987) are used. In the following examples, when the score was 25.0 or higher, it was judged that the partial amino acid sequences are aligned (homologous) between a protein or polypeptide (A) and another protein or polypeptide (B).

The “m” amino acid partial sequences are premised to be aligned between a protein or polypeptide (A) and another protein or polypeptide (B) with their scores being Si (1≦i≦m) and the amino acid length of protein or polypeptide (B) being LB. The index for predicting the interaction of protein or polypeptide (A) with protein or polypeptide (B) calculated from the result of local alignment is defined as the sum (Si)/LB. It is predicted that the higher the index, the stronger the interaction.

### Example 4

### Prediction of Interaction of VTII with Bcl-2

Verotoxin 2 (VTII) of Escherichia coli 0157:H7 causes food poisoning and renal damage in human beings, but the mechanism of action is not well-known (Sandvig, K. , et al., Exp. Med. Biol. 412, 225-232, 1997; Paton, J C., and Paton, A W. Clin. Microbiol. Rev. 11, 450-479, 1998). This protein is a toxic protein. Therefore, human proteins relating to cell death serve as candidates of proteins interacting with this protein. Thus, human proteins that may interact with this protein were searched, specifically for human proteins relating to cell death, in protein database SWISS-PROT version 35, and an example is given below showing that they actually interact with each other.

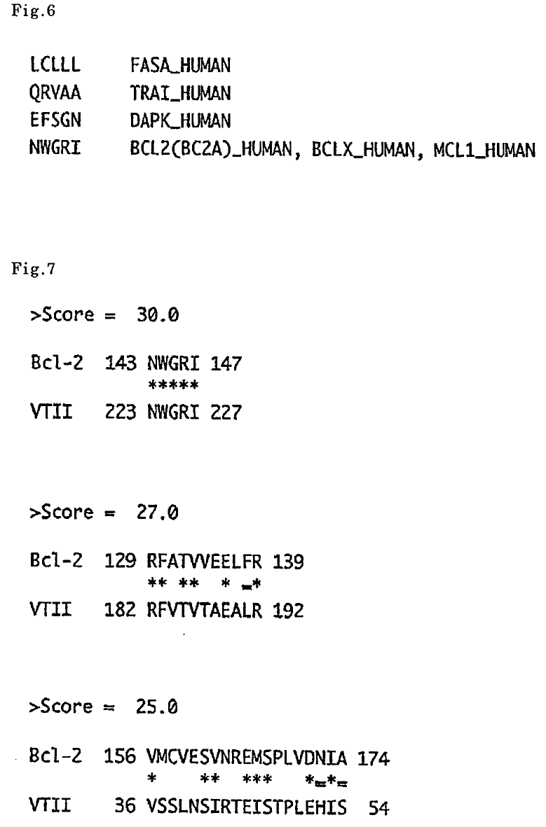

Among oligopeptides having an amino acid sequence length of 5 residues of verotoxin 2, those found to be contained in a human protein relating to cell death were the following four, i-e., LCLLL (SEQ ID NO: 5), QRVAA (SEQ ID NO: 6), EFSGN (SEQ ID NO: 7), and NWGRI (SEQ ID NO: 1), in SWISS-PROT version 35 (see FIG. 6, where the human proteins are shown by using protein IDs of SWISS-PROT version 35). Values of particularity for these oligopeptides were calculated from the amino acid frequencies in all of the proteins encoded by the genome of Escherichia coli shown in FIGS. 4a-b, i.e., the particularity of LCLLL (SEQ ID NO: 5) was 15001.03×10⁻¹⁰; that of QRVAA (SEQ ID NO: 6) was 15584.55×10⁻¹⁰; that of EFSGN (SEQ ID NO: 7) was 3801.65×10⁻¹⁰; that of NWGRI (SEQ ID NO: 1) was 1479.85×10⁻¹⁰. It was found that NWGRI (SEQ ID NO: 1) has the highest particularity among these four oligopeptides.

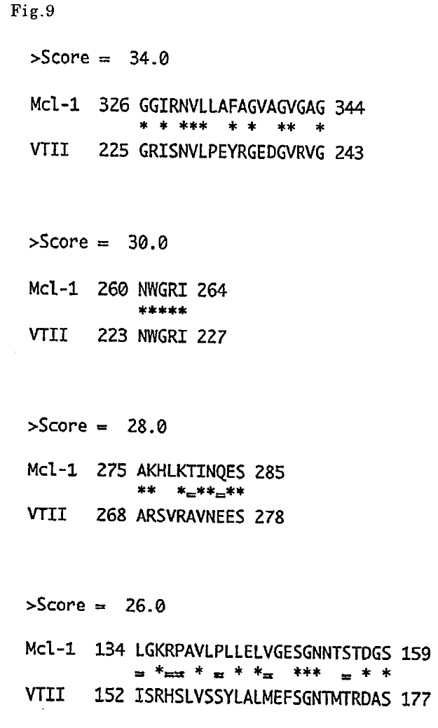

Oligopeptide NWGRI (SEQ ID NO: 1) comprises a portion of verotoxin 2 and each of three human proteins, i.e., Bcl-2, Bcl-xL, and MCL-1. Local alignment between verotoxin 2 (VTII) and each of Bcl-2, Bcl-xL, and MCL-1 revealed partial homology in their amino acid sequence, as shown in FIGS. 7, 8, and 9. Then, the sum of the scores of the local alignment was divided by the length of each protein to give index as described in Example 3, and shown below.

Bcl-2(30.0+27.0+25.0)/239=0.343

Bcl-xL(30.0+29.0+27.0)/233=0.369

MCL-1(34.0+30.0+28.0+26.0)/350=0.337

Among these three proteins, Bcl-2 and Bcl-xL constitutes the same family. Based on the index calculated from the local alignment by the above method, the prediction is that Bcl-2 and Bcl-xL have the highest interaction with verotoxin 2.

Verotoxin 1 (VTI) is one of the verotoxins produced by Escherichia coli O157:H7, and is an isoform of verotoxin 2. The toxicity of verotoxin 1 is weaker than that of verotoxin 2, with the former being about one fiftieth the latter (Tesh, V L., et al., 1993, Infect. Immun. 61, 3392-3402). In protein database SWISS-PROT version 35, a human protein that contains an oligopeptide having an amino acid length of 5 residues that comprises a portion of verotoxin 1 and is related to cell death is P2X1_HUMAN, the oligopeptide being SSTLG (SEQ ID NO: 8). However, the particularity of oligopeptide SSTLG (SEQ ID NO: 8), which is calculated to be 14385.63×10⁻¹⁰ from the amino acid frequencies in all of the proteins encoded by the genome of Escherichia coli shown in FIGS. 4a-b, is lower than that of NWGRI (SEQ ID NO: 1), by about one tenth.

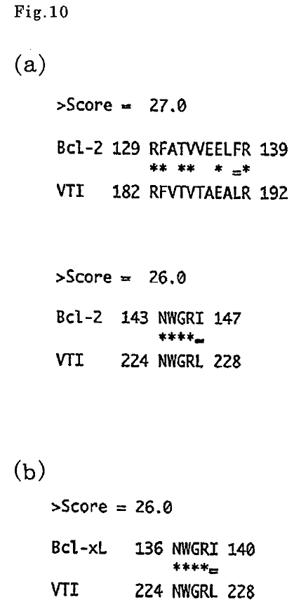

In verotoxin 1, the oligopeptide NWGRL (SEQ ID NO: 9) that corresponds to oligopeptide NWGRI (SEQ ID NO: 1) having an amino acid length of 5 residues in verotoxin 2 reveals a particularity of 2622.30×10⁻¹⁰, calculated from FIGS. 4a-b, that is lower than that of NWGRI (SEQ ID NO: 1). Both Bcl-2 and Bcl-xL contain oligopeptide NWGR (SEQ ID NO: 10) having an amino acid sequence length of 4 residues that comprises a portion of verotoxin 1. Comparison between the particularity of NWGRI (SEQ ID NO: 1) and that of NWGRL (SEQ ID NO: 9) permits prediction that both Bcl-2 and Bcl-xL interact more strongly with verotoxin 2 than with verotoxin 1. In addition, the indexes obtained by the calculation from the result (FIG. 10) of the local alignment between verotoxin 1 and Bcl-2 or Bcl-xL are (27.0+26.0)/239=0.222 and 26.0/233=0.112, respectively (there is no homologous amino acid partial sequence other than the NWGR part). Consequently, it is predicted that the interaction of verotoxin 1 with Bcl2 or Bcl-xL is considerably weaker than the interaction of verotoxin 2 with Bcl-2 or Bcl-xL.

### Example 5

### Experimental Confirmation of Prediction of Interaction of VTII with Bcl-2

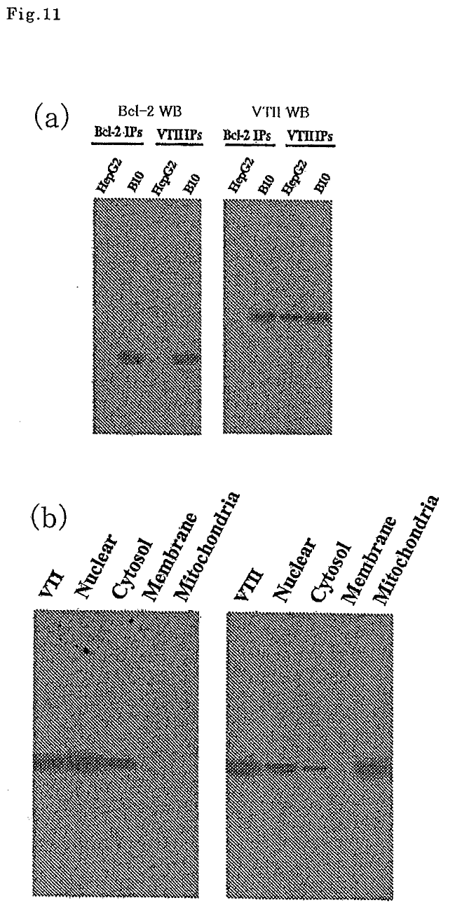

In Example 4, the reliability of prediction that verotoxin 2 interacts with human Bcl-2 or Bcl-xL was predicted to be high. Based on the result of this prediction, it was experimentally confirmed that verotoxin 2 actually interacts with Bcl-2 (FIG. 11a and FIG. 11b (right)). Specifically, human hepatic cancer cell HepG2 (essentially not expressing the Bcl-2 gene) and B10 cells prepared by transducing a Bcl-2-expressing vector into HepG2 so as to express Bcl-2, were treated with verotoxin 2 (VTII), and then co-immunoprecipitation was conventionally carried out using anti-Bcl-2 antibody (Bcl-2 IPs) and anti-VTII antibody (VTII IPs).

FIG. 11a (left) illustrates the result of the western blotting analysis using anti-Bcl-2 antibody after co-immunoprecipitation; FIG. 11a (right) illustrates the result of the western blotting analysis using anti-VTII antibody. It was confirmed from these results that a VTII/Bcl-2 complex was co-immunoprecipitated in the B10 cells, i.e., these two proteins interact with each other. Moreover, B10 cells were treated with verotoxin 1 (VTI) or verotoxin 2 (VTII) to examine in which subcellular fraction these proteins were detected using anti-VTI antibody and anti-VTII antibody. Bcl-2 in mitochondria plays a very important role in cell death. Verotoxin 2 (VTII) was detected also in a mitochondria fraction (FIG. 11b (right)).

On the other hand, verotoxin 1 was not detected in the mitochondria fraction. Thus, it was proved experimentally that verotoxin 1 does not have a strong interaction with mitochondria Bcl-2. The result is shown in FIG. 11b (left)

### Example 6

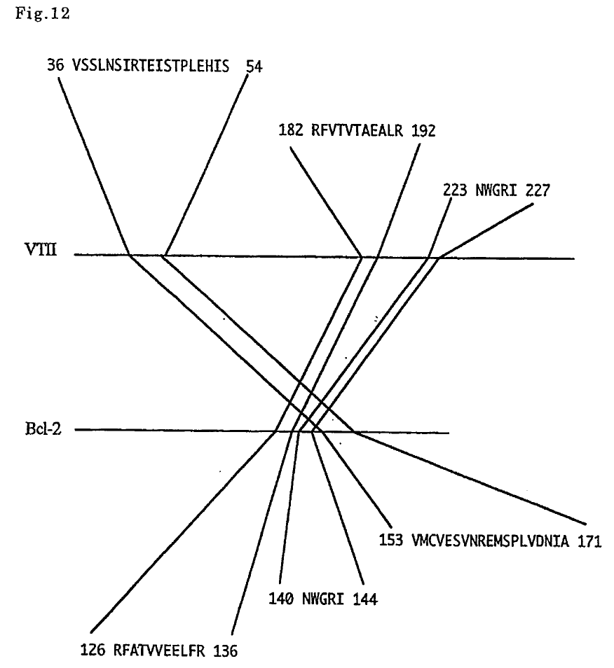

FIG. 12 illustrates an example wherein the full-length amino acid sequences of verotoxin 2 (VTII) and Bcl-2 were displayed so as to show the locations of the partial sequences aligned in the full-length sequences.

### Example 7

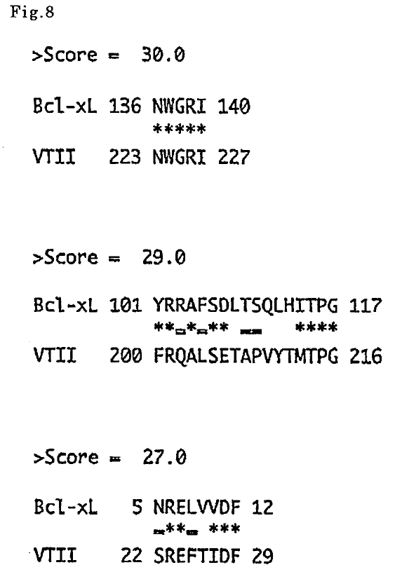

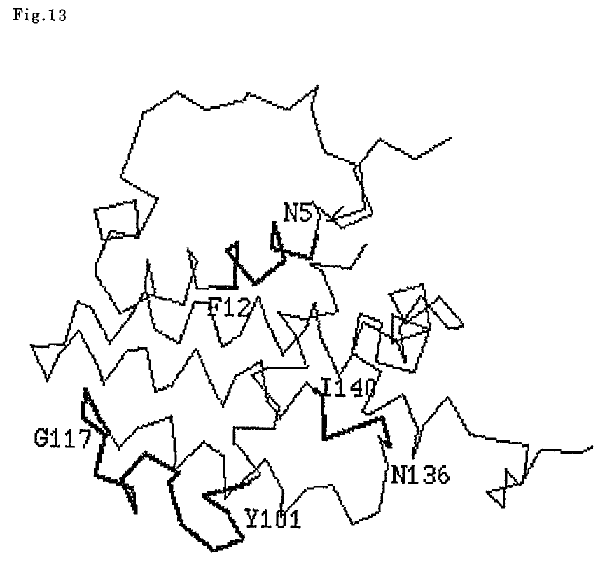

The stereo structure of Bcl-xL is known, with the structure being registered in PDB, that is a protein stereo structure database. Based on the result of the local alignment of FIG. 8, partial amino acid sequences homologous to those of verotoxin 2 in the stereo structure of Bcl-xL are shown with bold lines in FIG. 13.

### Example 8

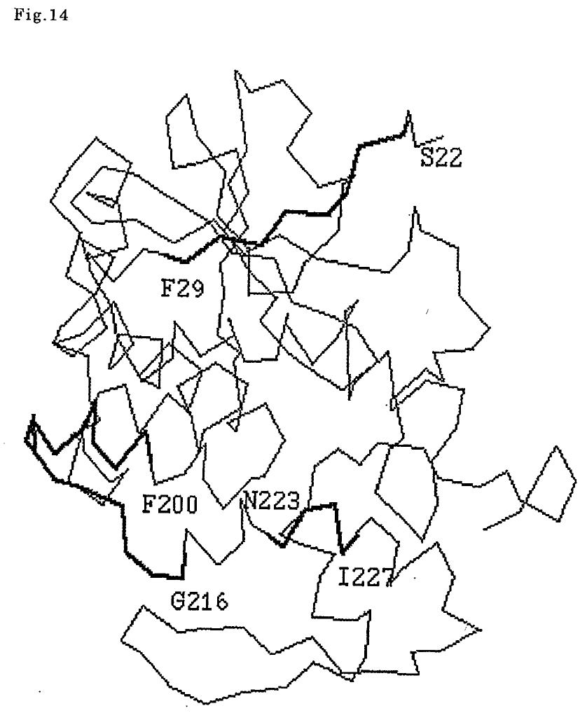

Verotoxin 2 is believed to cleave a part of ribosomal RNA so as to stop protein synthesis, thereby exerting its toxicity. The stereo structure of protein ‘ricin’ that cleaves a part of ribosomal RNA is registered in PDB, that is a protein stereo structure database. Based on the structure, homology modeling of verotoxin 2 was carried out. Based on the result of the local alignment of FIG. 8, the amino acid partial sequences homologous to those of Bcl-xL is shown in the stereo structure model with bold lines in FIG. 14.

### Example 9

### Suppression by NWGRI of Cell Death Induction by VTII

Next, it was experimentally confirmed that oligopeptide NWGRI (SEQ ID NO: 1), which was found in Example 4 and comprises a portion of VTII and Bcl-2, can control the interaction of VTII with Bcl-2. First of all, the complex formation was examined using an extract of the Bcl-2-expressing B10 cells used in Example 5 and biotinylated VTII in the presence of oligopeptide NWGRI (SEQ ID NO: 1), and then analyzed by Far Western blotting analysis. Oligopeptide NWGRI (SEQ ID NO: 1) interrupted the complex formation of VTII and Bcl-2 in a dose dependent manner.

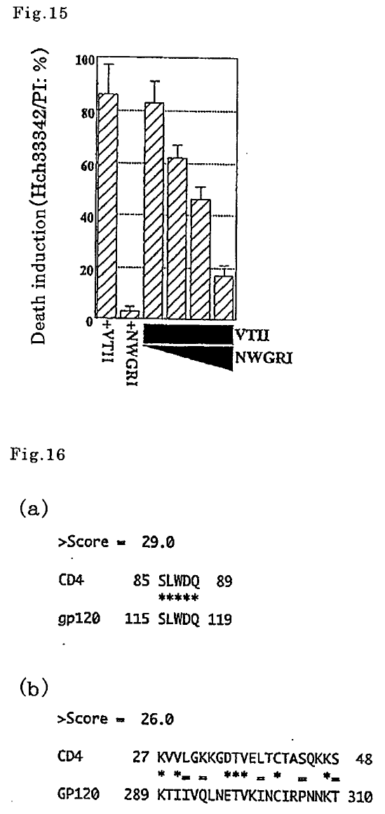

In addition, B10 cells were pretreated with oligopeptide NWGRI (SEQ ID NO: 1) at 0, 10, 50, 100 μM and were treated with VTII at 10 ng/ml for 24 hr, and the induction of cell death by apoptosis was assayed. A total of about 5,000 nuclei was dyed with Hoechst 33342/PI (Propidium iodide) according to the manufacturer's instruction, and the ratio of nuclei that showed apoptosis is shown in FIG. 15. As shown in the figure, about 85% of cells caused apoptotic cell death by the treatment with only VTII, while the induction of apoptotic cell death was suppressed by pretreatment with oligopeptide NWGRI (SEQ ID NO: 1) in a dose dependent manner. Thus, it was confirmed that oligopeptide NWGRI (SEQ ID NO: 1), which comprises a portion of VTII and Bcl-2, interrupts the interaction of VTII with Bcl-2 so as to inhibit the complex formation of these proteins and suppresses cell death induction by VTII thereupon.

### Example 10

### CD4/gp120HIV-1

Human AIDS virus HIV-1 infects helper T cells. An important first step to infecting these cells is that protein gp120 on the viral surface binds to surface protein CD4 of helper T cells. In this example, it was examined if the binding of gp120 and CD4 can be predicted by the above prediction method.

Protein CD4 was decomposed into oligopeptides having an amino acid sequence length of 5 resides, and proteins having the amino acid sequence of the oligopeptide derived from CD4 were serially searched in a protein database, and gp120 was extracted as a protein that contains oligopeptide SLWDQ (SEQ ID NO: 11) (FIG. 16a). Oligopeptide SLWDQ (SEQ ID NO: 11) exists only in protein CD4 as a human protein in SWISS-PROT version 35, i.e., the frequency in human proteins is 1 and the particularity is very high. Moreover, besides this oligopeptide, a locally homologous region exists (FIG. 16b). It is known that amino acid residue arginine (Arg) next to oligopeptide SLWDQ (SEQ ID NO: 11) in the N-terminal side and 67-SFLTKGP-73 (SEQ ID NO: 12) play important roles when CD4 binds to gp120 (Kwong, PD., et al., Nature, vol. 398, 648-659, 1998). It is also known that a few amino acid residues next to the homologous region (289-KTIIVQLNETVKINCIRPNNKT-310) (SEQ ID NO: 13) shown in FIG. 16b in the N-terminal side is one of the regions playing an important role when CD4 is recognized by gp120 (Kwong, PD., et al., Nature, vol. 398, 648-659, 1998). Therefore, even if the binding between gp120 and CD4 is not known, it can be predicted by the above prediction method.

### Example 11

### CED-4/MAC-1

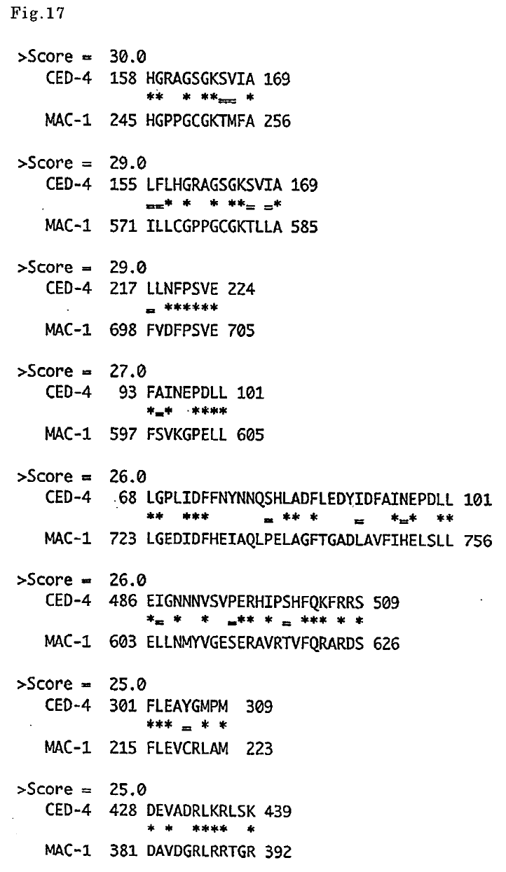

Nematode Caenorhabditis elegans is the first multicellular organism whose entire genome information was elucidated. One example concerning C. elegans is described here. Protein CED-4 plays a central role in the control of programmed cell death. MAC-1 was found to be a protein that binds to CED-4 and suppresses cell death (Wu et al., Development, vol. 126, 9, 2021-2031, 1999). Therefore, oligopeptides that comprise a portion of these two proteins were examined so as to verify the present invention, although the binding between MAC-1 and CED-4 is known. As a result, it was found that MAC-1 and CED-4 contain the same oligopeptide FPSVE (SEQ ID NO: 14) having an amino acid sequence length of 5 residues, and the present invention was verified. The index of this oligopeptide, calculated from a frequency of amino acids in the genome of C. elegans, was 5.436. Moreover, as illustrated in FIG. 17, there are many homologous regions between these two proteins, whereby the binding of these proteins was strongly suggested (top sequence, CED-4; bottom sequence, MAC-1).

### Example 12

### APP/BASE

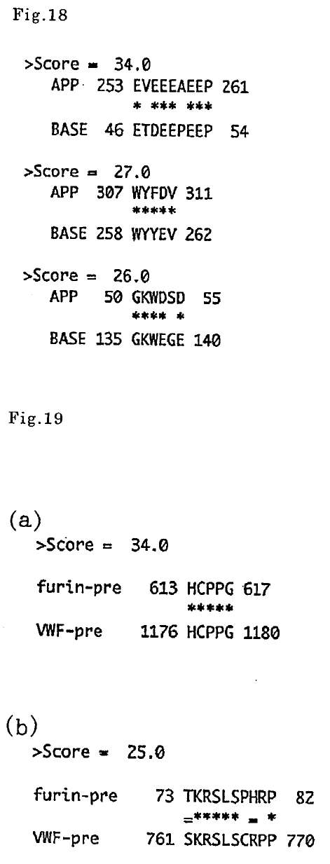

APP (amyloid precursor protein), which is one of the proteins causing Alzheimer's disease, gives rise to amyloid upon being cleaved at two sites. An enzyme (BASE, bata secretase) that cleaves the site on the amino terminal side of the two cleavage sites was recently discovered (VASSAR et al., Science, 286(5440), 735-741, 1999). Cleavage of APP by BASE indicates the presence of the interaction of these two proteins. To verify the present invention, oligopeptides that comprise a portion of these two proteins were examined. APP and BASE have homologous oligopeptides WYFDV (SEQ ID NO: 15) and WYYEV (SEQ ID NO: 16) having an amino acid sequence length of 5 residues that comprise a portion of each of them. The oligopeptide WYFDV (SEQ ID NO: 15) exists only in protein APP as a human protein in SWISS-PROT version 35. A human protein comprising WYYEV (SEQ ID NO: 16) is not registered yet. Both oligopeptides have high particularity. This result verified the present invention. FIG. 18 illustrates the regions homologous between the two proteins (top sequence, APP; bottom sequence, BASE).

### Example 13

### Furin and Von Willebrand Factor

Furin is an intracellular serine protease, and is related to the secretion system pathway, such as von Willebrand factor (VWF), albumin, and complement C3. An example of the interaction of furin with VWF is mentioned here. VWF is cleaved from a precursor protein by furin to act as a mature protein. Cleavage of the VWF precursor protein by furin requires the interaction of these two proteins. Moreover, furin per se becomes a mature protein from a precursor protein of furin by being cleaved to act as a protease. Therefore, to verify the present invention, an oligopeptide that comprises a portion of furin precursor protein and VWF precursor protein was examined. The two proteins comprising the same oligopeptide HCPPG (SEQ ID NO: 17), at positions 613-617 of furin precursor protein and at positions 1176-1180 of VWF precursor protein (FIG. 19a). Both locations are within the regions of the mature proteins. The oligopeptide HCPPG (SEQ ID NO: 17) comprises a portion of only furin precursor protein and VWF precursor protein as human proteins in SWISS-PROT version 35, and has high particularity from the viewpoint of frequency. VWF precursor protein is cleaved by furin at the site between the 763rd amino acid residue and the 764th amino acid residue. Local alignment between furin precursor protein and VWF precursor protein reveals that the region near the site of VWF precursor protein cleaved by furin has a partial region homologous to furin precursor protein (FIG. 19b). Thus, even if a novel protein was presumed to be a protease by the motif of the active part, and a counterpart protein and/or the cleavage site in the counter protein were not known, the present invention permits predicting the counterpart protein, as well as the cleavage site in the counter protein.

### Example 14

### APP and PC7

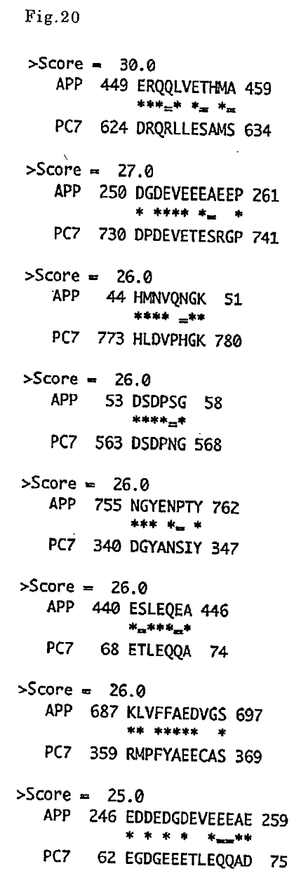

APP alpha is a peptide formed by cleavage of amyloid precursor protein (APP) at a site different from the two cleavage sites to form amyloid. It was recently found that PC7 (proprotein convertase subtilisin/kexin type 7) is involved in cleavage for forming APP alpha (Lopez-Perez E et al., J. Neurochem., vol. 73, 5, 2056-2062, 1999). Examination of the oligopeptide that comprises a portion of the two proteins APP and PC7 revealed that APP and PC7 have homologous oligopeptides DSDPSG (SEQ ID NO: 18) and DSDPNG (SEQ ID NO: 19) having an amino acid sequence length of 6 residues that comprise a portion of both of them. The oligopeptide DSDPSG (SEQ ID NO: 18) exists only in protein APP as a human protein in SWISS-PROT version 35. A human protein comprising DSDPNG (SEQ ID NO: 19) is not registered yet. Both oligopeptides have very high particularity. This result verified the present invention. FIG. 20 illustrates regions homologous between the two proteins.

Between K and L of 687-KLVFFAEDVGS-697 (SEQ ID NO: 20) of APP in FIG. 20 is the cleavage site to form APP alpha, and FIG. 20 illustrates that a partial sequence (359-RMPFYAEECAS-369) (SEQ ID NO: 21) homologous to this exists in PC7. Namely, this example shows that the present invention permits predicting a protein involved in cleaving another protein.

### Industrial Applicability

As described above in detail, the present invention permits predicting, by using a protein database, a counterpart protein that interacts with a protein having an unknown function that is obtained by genome analysis or cDNA analysis or a protein having a known function. Namely, the protein-protein interaction in one organism whose genome information was elucidated can be predicted on a computer using a protein database based on genome analysis and cDNA analysis that have been recently enhanced. If the prediction on a computer becomes possible, information concerning proteins that were predicted to interact with each other based on the prediction on a computer can be easily obtained without adopting a risky technique wherein the result depends on a cDNA library used, such as the two-hybrid method. The prediction became possible, so that it becomes possible to easily predict the sequence of an oligopeptide involved in the interaction, and to design a novel compound capable of controlling protein-protein interactions based on the information. The present invention makes elucidating protein-protein interactions efficient, and can be widely utilized in various fields including biochemistry, molecular biology, pharmaceutical development, agriculture, and biotechnology. Especially, in the development of pharmaceuticals, the present invention permits predicting the mechanism of disease that has not so far been known, and gives a possibility of creating novel pharmaceuticals.

What is claimed is:

1. A method for predicting whether a protein or polypeptide (B) interacts with a specific protein or polypeptide (A), wherein the method comprises the steps of:
1) decomposing the amino acid sequence of protein or polypeptide (A) into a series of oligopeptides having a pre-determined length, by shifting, serially, by one amino acid residue from the N-terminal end to the C-terminal end of said protein or polypeptide (A);
2) searching, within a selected database of protein or polypeptide amino acid sequences, for a protein or polypeptide (C) comprising an amino acid sequence for each member of the series or for a protein or polypeptide (D) comprising an amino acid sequence homologous to an amino acid sequence for each member of the series, and detecting said protein or polypeptide (C) or said protein or polypeptide (D);
3) carrying out local amino acid sequence alignment between said protein or polypeptide (A) and the detected protein or polypeptide (C) or detected protein or polypeptide (D), whereupon if the local amino acid sequence alignment gives rise to any homology between the protein or polypeptide (A) and the detected protein or polypeptide (C) and/or protein or polypeptide (D), then;
4) determining the frequency of each member of said oligopeptides in a database of all proteins encoded by the organism, from which said protein or polypeptide (A) is derived, by calculating a product of the frequency of each amino acids composing said member of the oligopeptides in said database, and/or determining the local amino acid sequence alignment score on said oligopeptides so as to calculate the value by dividing the sum of the scores by the amino acid length of the protein or polypeptide (C) or protein or polypeptide (D), whereby said frequency of said oligopeptide and/or said value indicates that said protein or polypeptide (C) or protein or polypeptide (D) corresponds to said protein or polypeptide (B) which interacts with said protein or polypeptide (A).

2. The method according to claim 1, wherein each oligopeptide is 4-15 amino acids in length.

3. A recording medium carrying a program to enable a computer system to predict whether a protein or polypeptide (B) interacts with a specific protein or polypeptide (A), comprising:
a) a means for inputting amino acid sequence information of the protein or polypeptide (A) and storing the information;
b) a means for decomposing said information into a series of oligopeptides having a pre-determined length as sequence information, and a means for storing the sequence information consequently obtained;
c) a means for storing an input protein database;
d) a means for accessing the stored protein database and detecting a protein or polypeptide (C) having an amino acid sequence of said oligopeptide or a protein or polypeptide (D) having an amino acid sequence homologous to the amino acid sequence of said oligopeptide, and a means for storing and calculating a detected result;
e) a means for carrying out local alignment between said protein or polypeptide (A) and the detected protein or polypeptide (C) or protein or polypeptide (D), and a means for storing and calculating a result; and
f) a means for obtaining a resultant value of a frequency of an amino acid and/or a frequency of said oligopeptide from a protein database, followed by showing an index for predicting protein-protein interactions from the resultant value and a resultant value of said local alignment, and a means for storing and displaying the result and consequently detecting B which interacts with the protein or polypeptide (A).

4. A recording medium characterized by comprising at least one or more of the following means in addition to the means according to claim 3:
g) a means for ranking strength of protein-protein interactions among detected proteins or polypeptides (B) based on the indexes calculated from a resultant value of local alignment and a resultant value of a frequency of an amino acid and/or a frequency of an oligopeptide in a protein database in the case that more than one protein or polypeptide (B) exist that are detected, and a means for storing and displaying the result;
h) a means for displaying full-length of amino acid sequences of said protein or polypeptide (A) and said protein or polypeptide (B) that is detected, followed by indicating a location of partial sequence to be aligned in the full-length sequence in the case that amino acid partial sequences are aligned by local alignment between the protein or polypeptide (A) and the protein or polypeptide (B);
i) a means for calculating a stereo structure model in the case that a stereo structure of said protein or polypeptide (A) or said protein or polypeptide (B) that is detected is known or in the case that homology modeling enables to make a stereo structure model, followed by displaying the structure of the amino acid partial sequences that are aligned by local alignment between the protein or polypeptide (A) and the protein or polypeptide (B) on the stereo structure;
j) a means providing a function of classifying and storing proteins in a protein database to narrow a searching area;
k) a means for serially inputting each protein in a protein database as said protein or polypeptide (A);
l) a means for storing a genome database.

5. A device for predicting protein-protein interactions, wherein the device comprises means which are carried by the recording medium according to claim 3 or 4.

6. A method which comprises identifying a protein or polypeptide (B) that is predicted to interact with a specific protein or polypeptide (A) using the method according to claim 1 or 2, and then experimentally confirming that said protein or polypeptide (A) interacts with said protein or polypeptide (B).

7. A method which comprises identifying a protein or polypeptide (B) that is predicted to interact with a specific protein or polypeptide (A) using the device according to claim 5, and then experimentally confirming that said protein or polypeptide (A) interacts with said protein or polypeptide (B).

8. An isolated protein or polypeptide identified by the method according to claim 6 or 7.

9. A method of screening for a compound that controls the interaction of a specific protein or polypeptide (A) with a protein or polypeptide (B), wherein the method utilizes the method according to claim 1 or 2.

10. A method of screening for a compound that controls the interaction of a specific protein or polypeptide (A) with a protein or polypeptide (B), wherein the method comprises the steps of:
1) contacting the compound with the protein or polypeptide (A) and the protein or polypeptide (B) that are predicted to interact with each other using the method according to claim 1 or 2, then;
2) detecting the presence or absence or change of the interaction using the system for detecting the interaction of protein or polypeptide (A) with the protein or polypeptide (B).

11. A method of screening for a compound that controls the interaction of a specific protein or polypeptide (A) with a protein or polypeptide (B), wherein the method uses the device according to claim 5.

12. A method of screening for a compound that controls the interaction of a specific protein or polypeptide (A) with a protein or polypeptide (B), wherein the method comprises the steps of:
1) contacting the compound with the protein or polypeptide (A) and the protein or polypeptide (B) that are predicted to interact with each other using the device according to claim 5, then;
2) detecting the presence or absence or change of the interaction using the system for detecting the interaction of protein or polypeptide (A) with the protein or polypeptide (B).

13. A compound obtained by the screening method according to claim 9.

14. A compound obtained by the screening method according to claim 10.

15. A compound obtained by the screening method according to claim 11.

16. A compound obtained by the screening method according to claim 12.

17. A compound which controls the interaction of a specific protein or polypeptide (A) with a protein or polypeptide (B), wherein the compound is obtained by drug design based on information of a compound obtained by the screening method according to claim 9.

18. A compound which controls the interaction of a specific protein or polypeptide (A) with a protein or polypeptide (B), wherein the compound is obtained by drug design based on information of a compound obtained by the screening method according to claim 10.

19. A compound which controls the interaction of a specific protein or polypeptide (A) with a protein or polypeptide (B), wherein the compound is obtained by drug design based on information of a compound obtained by the screening method according to claim 11.

20. A compound which controls the interaction of a specific protein or polypeptide (A) with a protein or polypeptide (B), wherein the compound is obtained by drug design based on information of a compound obtained by the screening method according to claim 12.

21. An oligopeptide comprising the amino acid sequence of SEQ ID No: 1, wherein the oligopeptide controls the interaction of verotoxin 2 (VTII) with Bcl-2.

22. A composition useful for inhibiting cell death comprising an oligopeptide comprising the amino acid sequence of SEQ ID NO: 1, and a pharmaceutically acceptable carrier or diluent.

23. An oligopeptide comprising an amino acid sequence homologous to the amino acid sequence of SEQ ID No: 1, wherein the oligopeptide controls the interaction of VTII with Bcl-2.

24. An isolated polypeptide comprising the amino acid sequence of the oligopeptide according to claim 21 or 23, wherein the polypeptide controls the interaction of VTII with Bcl-2.

25. A method of screening for a compound which controls the interaction of VTII with Bcl-2, wherein the method utilizes the oligopeptide according to claim 21 or 23

26. A method of screening for a compound which controls the interaction of VTII with Bcl-2, wherein the method comprises the steps of:
1) contacting the compound with VTII and the oligopeptide according to claim 21 or 23 or with Bcl-2 and the oligopeptide, then;
2) detecting the presence or absence or change of the interaction of oligopeptide with VTII or Bcl-2 using the system for detecting the interaction.

27. A method of screening for a compound which controls the interaction of VTII with Bcl-2, wherein the method utilizes the polypeptide according to claim 24.

28. A method of screening for a compound which controls the interaction of VTII with Bcl-2, wherein the method comprises the steps of:
1) contacting the compound with VTII and the polypeptide according to claim 24 or with Bcl-2 and the polypeptide, then;
2) detecting the presence or absence or change of the interaction of polypeptide with VTII or Bcl-2 using the system for detecting the interaction.

29. A method for determining the nucleotide sequence of an oligonucleotide encoding an oligopeptide which is involved in the interaction of a specific protein or polypeptide (A) with a protein or polypeptide (B), wherein the method uses the method according to claim 1 or 2 or the device according to claim 5.

30. A series of combinations of human proteins that are predicted to interact with each other, obtained by the method according to claim 1 or 2 or the device according to claim 5.

31. A method for selecting a combination of proteins which interact with each other, wherein said interaction is related to a disease, wherein the method comprises selecting the combination based on the information of a known protein that can be related to the disease from the series of combinations according to claim 30.

32. A series of combinations of proteins which interact with each other, wherein said interaction is related to a disease, wherein each member of said series is selected according to the method of claim 31.

33. A method of screening for a compound which controls the interaction of a combination of proteins selected from the series of combinations according to claim 32.

34. A compound obtained by the method according to claim 33.

35. A method for predicting a processing site of a protein comprising predicting the interaction of a specific protein with an enzyme cleaving said protein using the method according to claim 1 or 2 or the device according to claim 5.

36. An isolated polypeptide comprising an amino acid sequence that is predicted to contain a protein-processing site obtained by the method according to claim 35 and/or to contain a partial sequence homologous to the processing site.
